(19) United States
(12) Patent Application Publication
(10) Pub. No.: US 2011/0272337 A1
(43) Pub. Date: Nov. 10, 2011

(54) DUAL MODE HEMODIALYSIS MACHINE

(75) Inventor: Grant Palmer, Burbank, CA (US)

(73) Assignee: C-TECH BIOMEDICAL, INC., Anaheim, CA (US)

(21) Appl. No.: 13/100,847

(22) Filed: May 4, 2011

Related U.S. Application Data
(60) Provisional application No. 61/331,273, filed on May 4, 2010.

(51) Int. Cl. A61M 1/34

(52) U.S. Cl. 210/195.2

(57) ABSTRACT

A compact portable dual mode hemodialysis machine system is provided. The system includes a sorbent dialysis module with a sorbent cartridge that purifies a dialysate fluid that flows therethrough, where the sorbent dialysis module returns the purified dialysate fluid from the sorbent cartridge to an inlet of a dialyzer. The system also includes a single-pass dialysis module with an acetate pump, a bicarbonate pump and a mixing chamber, where the acetate and bicarbonate pumps flow acetate and bicarbonate mixtures, respectively, into the mixing chamber. The single-pass dialysis module receives a desired amount of water from a reverse osmosis device, the single-pass dialysis module operated to direct used dialysate from the dialyzer to a drain. The machine system can be operated to replace the sorbent cartridge with the single-pass dialysis module to switch the operation of the dual mode hemodialysis machine system from a sorbent dialysis mode to a single-pass dialysis mode

### CROSS-REFERENCE TO RELATED APPLICATIONS

This application claims the benefit of U.S. Provisional Patent Application No. 61/331,273 filed May 4, 2010, the entire contents of which are incorporated herein by reference and should be considered a part of this specification

### BACKGROUND

1. Field

The present invention is directed to hemodialysis machines, and more specifically to a dual mode portable hemodialysis machine.

2. Description of the Related Art

Conventional single-pass hemodialysis machines are bulky and difficult to handle by medical personnel who operate the machines to conduct dialysis treatments on patients. Additionally, such single-pass machines require a dedicated source of water, and water treatment system, and so are generally used in hospitals and dialysis clinics. Patients who visit such clinics or hospitals for dialysis treatments can spend up to 6 hours at such facilities per treatment, which is a great source of inconvenience for busy individuals and can cause great disruptions to work and family life, as well as travel plans. Because of the need for a dedicated water source and their bulkiness and cost, such single-pass machines are not mobile and generally in a fixed location within the hospital or dialysis clinic.

Existing sorbent hemodialysis machines, though they are generally portable and to not need a dedicated water source, suffer from several deficiencies. One such deficiency is the low dialysate flows provided by the sorbent hemodialysis machines. Such lower flows require patients to undertake more frequent treatments, not all of which may be fully reimbursable. Therefore, in addition to the added inconvenience of needing more frequent dialysis treatments, conventional sorbent hemodialysis machines can result in added expense to the patient.

A need exists for an improved hemodialysis machine that can be used both in a hospital or clinic setting, as well as in the home or while traveling, and that addresses the deficiencies noted above.

### SUMMARY

In accordance with one embodiment, a compact portable dual mode hemodialysis machine system is provided. The system comprises a sorbent dialysis module comprising a sorbent cartridge configured to purify a dialysate fluid that flows therethrough. The sorbent dialysis module is configured to return a purified dialysate fluid from the sorbent cartridge to an inlet of a dialyzer. The system also comprises a single-pass dialysis module comprising an acetate pump, a bicarbonate pump and a mixing chamber, the acetate and bicarbonate pumps configured to flow an acetate and bicarbonate into the mixing chamber. The single-pass dialysis module is further configured to receive a desired amount of water from a reverse osmosis device, the single-pass dialysis module configured to direct used dialysate from the dialyzer to a drain. The single-pass dialysis module is configured to replace the sorbent cartridge to switch the operation of the dual mode hemodialysis machine system from a sorbent dialysis mode to a single-pass dialysis mode

### BRIEF DESCRIPTION OF THE DRAWINGS

FIG. 1A is a schematic perspective top view of one embodiment of a dual mode hemodialysis machine.

FIG. 1B is a schematic perspective rear view of the dual mode hemodialysis machine of FIG. 1A.

FIG. 2 is a schematic flow diagram of components of another embodiment of a dual mode hemodialysis machine.

FIG. 3A is a schematic sectional view of one portion of a flow diagram of another embodiment of a dual mode hemodialysis machine.

FIG. 3B is a schematic sectional view of one portion of a flow diagram of another embodiment of a dual mode hemodialysis machine.

FIG. 4 is a schematic front view of one embodiment of a sorbent cartridge for use with a dual mode hemodialysis machine.

FIG. 5 is a schematic flow diagram of a blood flow path of a dual mode hemodialysis machine.

FIG. 6A is a schematic flow diagram of a blood flow path of another embodiment of a dual mode hemodialysis machine.

FIG. 6B is a schematic sectional view of one portion of a blood flow path in another embodiment of a dual mode hemodialysis machine.

FIG. 7 is a schematic flow diagram of a single-pass module portion of another embodiment of a dual mode hemodialysis machine.

FIG. 8A is a schematic sectional view of one portion of a dialysate flow path in another embodiment of a dual mode hemodialysis machine.

FIG. 8B is a schematic sectional view of one portion of a dialysate flow path in another embodiment of a dual mode hemodialysis machine.

FIG. 9 is a schematic sectional view of one portion of a dialysate flow path in another embodiment of a dual mode hemodialysis machine.

### DETAILED DESCRIPTION

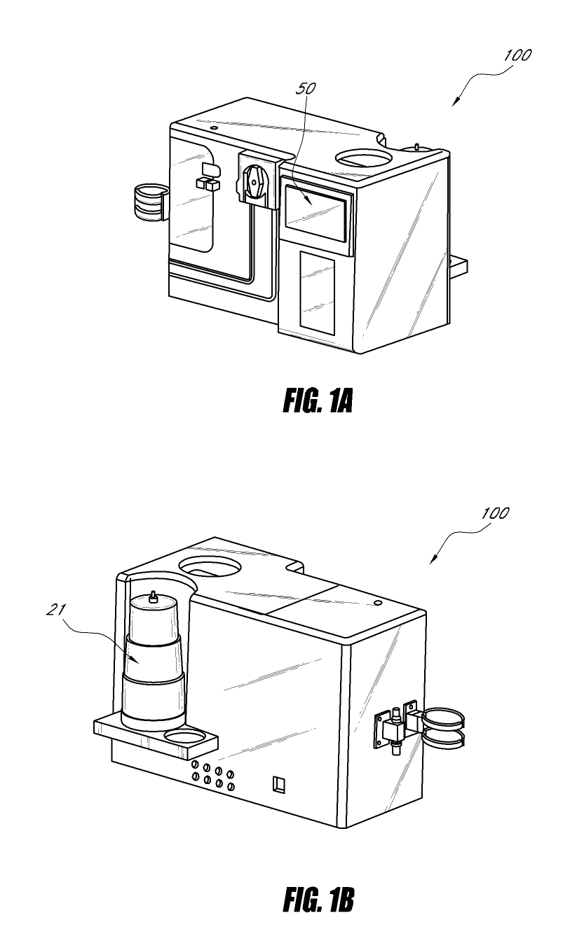

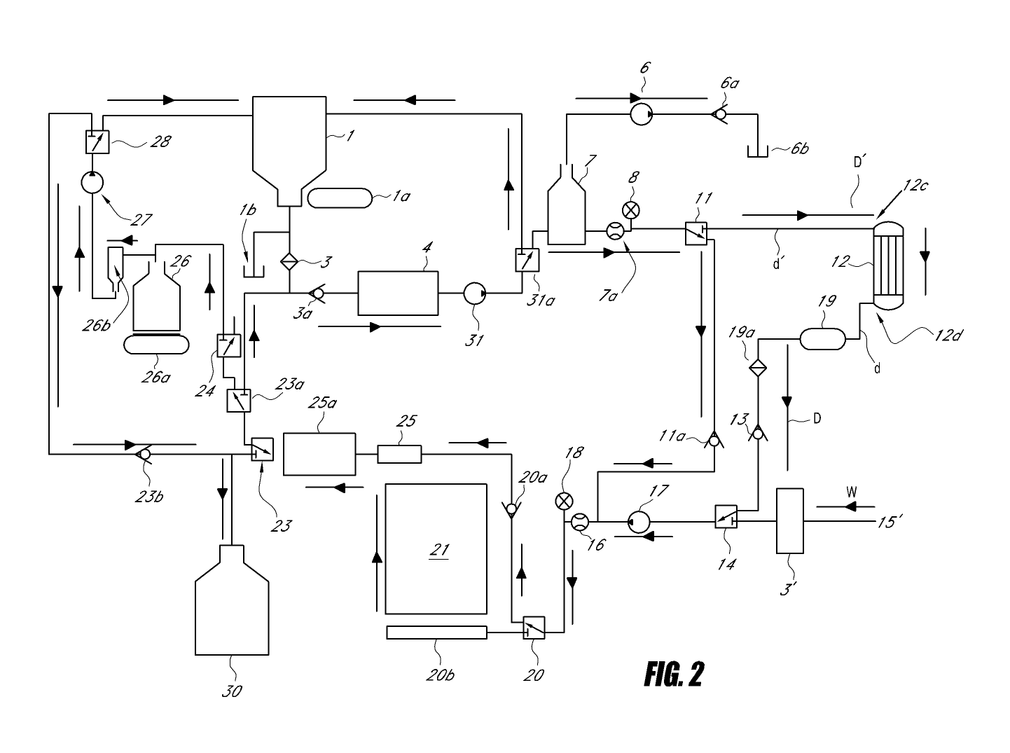

FIGS. 1A and 1B show one embodiment of a dual mode hemodialysis machine 100. In one embodiment, the machine 100 is generally rectangular in shape and can have a length L, height H and depth D of about 2 feet by about 1 foot by about 1 foot. In another embodiment, the machine 100 can have dimensions of about 23 inches by about 12 inches by about 15 inches. In still another embodiment, the machine 100 can have length, width and depth dimensions of about 23 inches by 11 inches by 14 inches. In one embodiment, the machine 100 can advantageously weigh 60 lbs or less. In another embodiment, the machine 100 can weigh 50 lbs or less. In still another embodiment, the machine can weigh about 45 lbs.

With continued reference to FIGS. 1A and 1B, the dual mode hemodialysis machine 100 can include a sorbent cartridge 21 removably coupleable to a base of the machine 100. The machine 100 can also have a screen 50, such as a digital video screen, for displaying various parameters of the operation of the machine 100.

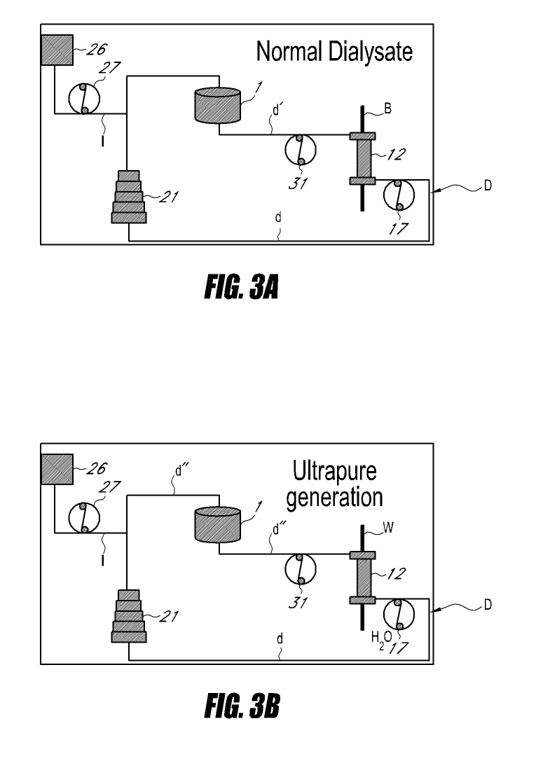

FIG. 2 shows an embodiment of a dialysate flow path D′ of a dual mode hemodialysis machine, such as the hemodialysis machine 100.

In the illustrated embodiment, unclean dialysate d exits the dialysate outlet 12d of the dialyzer 12 and through a blood leak detector 19, in-line filter 19a, check valve 13 and rinse/fill valve 14. In one embodiment, the rinse/fill valve 14 can be actuated to allow flow of tap water W from a tap water source 15′ that passes through a filter 3′ (e.g., a carbon pre-filter). From the rinse/fill valve 14, the unclean dialysate d passes through a pump 17, past a flow meter 16 and pressure meter 18, and past a sorbent bypass valve 20. In one embodiment, the sorbet bypass valve 20 is actuated to allow the dialysate flow d to pass through an external base 20b and disposable sorbent canister or cartridge 21. In another embodiment, the sorbent bypass valve 20 can be actuated to bypass the sorbent cartridge 21 (e.g., to isolate the sorbent cartridge 21 to replace the cartridge 21). In the bypass mode, the dialysate flow d can flow past a check valve 20a. Downstream of the check valve 20a and sorbent canister 21, the clean dialysate flow d′ flows through a heater 25, temperature conductivity device 25a and power drain valve 23. The power drain valve 23 can be actuated to allow flow of clean dialysate d′ into a drain container 30. From the power drain valve 23, the dialysate d′ flow can flow through a reservoir bypass valve 23a, infusate fill valve 24 and into a reservoir 1 in communication with a load cell 1a.

An infusate I (e.g., bicarbonate) from an infusate reservoir 26 in communication with 26a flows through an infusate drip flow container 26b and infusate pump 27 and infusate power drain valve 28 to the reservoir 1. In one embodiment, the infusate power drain valve 28 can be actuated to direct flow the infusate I to the drain container 30 via check valve 23b. From the reservoir 1, the clean or purified dialysate d′ can flow through an in-line filter 3, check valve 3a, temperature conductivity device 4, pump 31 and dialysate mix valve 31. In one embodiment, the reservoir bypass valve 23a can be actuated to bypass the reservoir 1 and so the purified dialysate d′ instead flows to the check valve 3a. Downstream of the dialysate mix valve 31a, the purified dialysate d′ flows through a de-aeration chamber and regulator 7, past a flow sensor 7a and pressure sensor 8, through a dialyzer bypass valve 11 and into a dialysate inlet 12c of the dialyzer 12. In one embodiment, the dialysate mix valve 31a can be actuated to recirculate the purified dialysate d′ flow back to the reservoir 1. The de-aeration chamber and regulator 7 is in communication with a vacuum pump 6 and check valve 6a that is in communication with a drain 6b, the de-aeration chamber 7 configured to remove gas from the dialysate d′ flow.

During operation, blood b with waste flows into a blood inlet 12a of the dialyzer 12 (as further discussed below), and clean or purified dialysate d′ enters a dialysate inlet 12c of the dialyzer 12, which can be disposed at an opposite end of the dialyzer 12 from the blood inlet 12a. As blood b and dialysate d′ flow through the dialyzer 12, waste products are removed from the blood b and transferred to the dialysate d. Clean blood b′ (e.g., blood from which waste products have been removed) exits a blood outlet 12b of the dialyzer 12 and is returned to the patient (as further discussed below), and unclean dialysate d exits the dialysate outlet 12d of the dialyzer 12 and is directed to the sorbent cartridge 21 to remove the waste therefrom, or to a drain when the hemodialysis machine 100 is operated in a single-pass mode, as further discussed below.

In one embodiment, the dual mode hemodialysis machine (e.g., the hemodialysis machine 100) can be operated in sorbent mode and single-pass mode, as further discussed below, which advantageously allows medical staff and user-patients to use the hemodialysis machine 100 in both manners. In another embodiment, the hemodialysis machine 100 can be used to provide peritoneal dialysis fluid, and can also be operated for automated rinse-back.

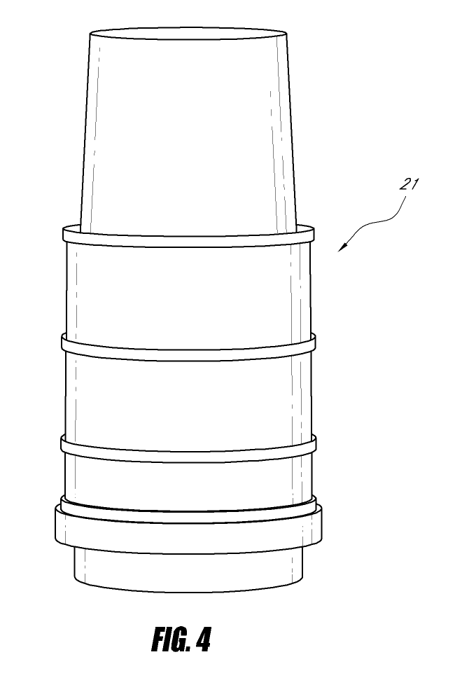

FIG. 3A shows a section of one embodiment of a dialysate flow path D of a dual mode hemodialysis machine, such as the hemodialysis machine 100. In the illustrated embodiment, dialysate d flows out of the dialyzer 12 via a pump 17 through the sorbent cartridge 21, where the dialysate is purified. As discussed above, the dialyzer 12 removes waste from blood B that flows therethrough. Purified dialysate d′ exits the sorbent cartridge 21 and flows to the dialysate reservoir 1 for storage. From the reservoir 1, the dialysate d′ is pumped via pump 31 to the dialysate inlet of the dialyzer 12. Infusate I from an infusate reservoir 26 is pumped via a pump 27 into the dialysate flow d′ exiting from the sorbent cartridge 21. In one embodiment, the pumps 31, 17 advantageously pump dialysate at a rate of between about 50 mL and about 600 mL (e.g., between 50 mL and 600 mL). In another embodiment, the pumps 31, 17 pump dialysate d, d′ at a rate of between about 250 mL and about 600 mL (e.g., between 250 mL and 600 mL). Advantageously, the use of pumps 17, 31 downstream and upstream of the dialyzer 12 facilitates the operation of the hemodialysis machine 100.

During operation of the hemodialysis machine 100, a user or medical personnel (e.g., nurse) can set the speed of the dialysate pump 31. During the dialysis treatment, a controller (further discussed below) can automatically adjust (e.g. increase or decrease) the speed of pump 17 to pull fluid from the patient via the dialyzer 12. In contrasts, conventional sorbent dialysis systems use only one pump downstream of the dialyzer, which pulls dialysate across the dialyzer. However, if the patient's body gives up more fluid, the dialysis machine does not automatically adjust speed to speed up or slow down the pump flow, which can result in disadvantageously removing too much fluid from a patient.

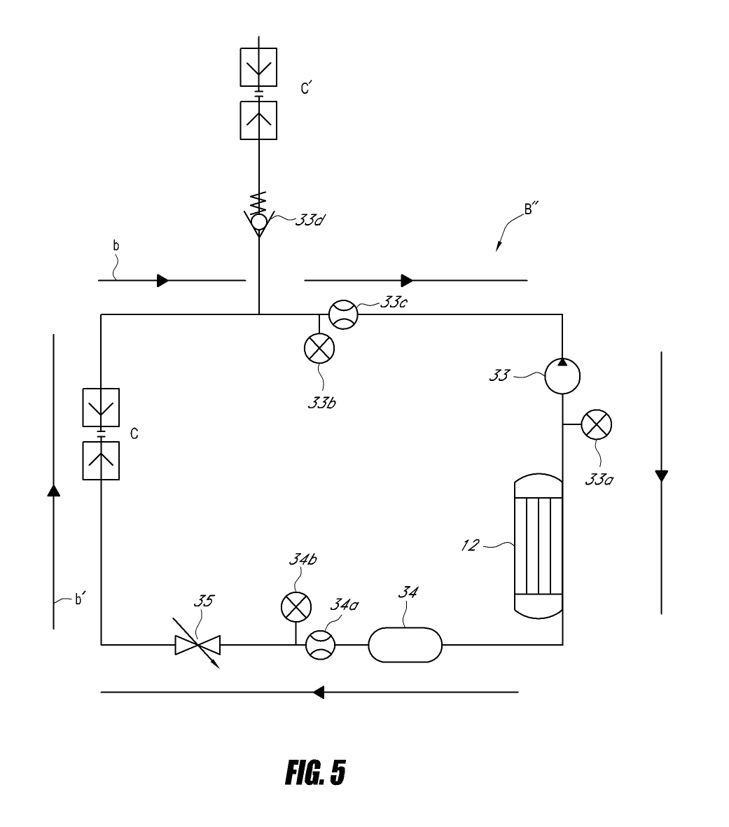

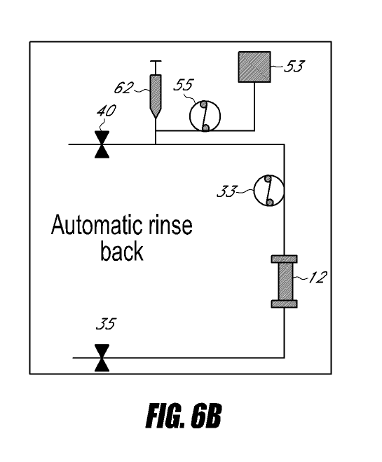

In one embodiment, the hemodialysis machine 100 can be used to generate ultrapure dialysate d″, as shown in FIG. 3B. In the illustrated embodiment, water W, not blood, is passed through the blood side of a dialyzer 12. Water flow may be controlled via the “blood” pump and the venous clamp 35 (see FIG. 6A). Through the use of differential positive displacement pumps 17, 31 on either side of the dialyzer 12, an amount of water is drawn across the membrane (not shown) of the dialyzer 12. The dialysis membrane removes bacteria and particulates from the water W. The water W then passes through a sorbent column or cartridge 21 for chemical purification. The sorbent column or cartridge 21 (see FIG. 4) can be generally cylindrical and contain simple activated carbon or may have additional ingredients to allow removal of other substances. In one embodiment, the sorbent column or cartridge 21 can also have amounts of zirconium phosphate (NaHZrP) or zirconium oxide (ZrO). In other embodiments, the sorbent cartridge 21 also removes endotoxins. In one embodiment, the sorbent cartridge 21 has a maximum pressure drop of about 1900 mm Hg during operation when dialysate d flows therethrough at a flow rate of about 450 mL/min. In one embodiment, the sorbent cartridge 21 an allow a maximum flowrate of about 450 mL/min therethrough. In one embodiment, additives (e.g., per prescription) may be added to the purified dialysate d″, either manually in the reservoir chamber 1 or via the infusate pump 27. Additionally, the fluid path D can be heated to kill any bacteria in the system 100.

With continued reference to FIG. 3B, the ultrapure dialysate fluid d″ can then be used as a peritoneal fluid. In one embodiment, the dialyzer 12 can be removed and connectors changed to connect the system 100 to a reservoir bag or directly to a peritoneal catheter. The system 100 can then act as a fluid generator or as a cycler. In the cycler mode, the patient could initiate the purification at any time or the machine 100 could be preprogrammed to fill and purify, then wait starting at any time, to be ready for patient use. As a cycler, the program can be scheduled to complete near the patients normal bed time.

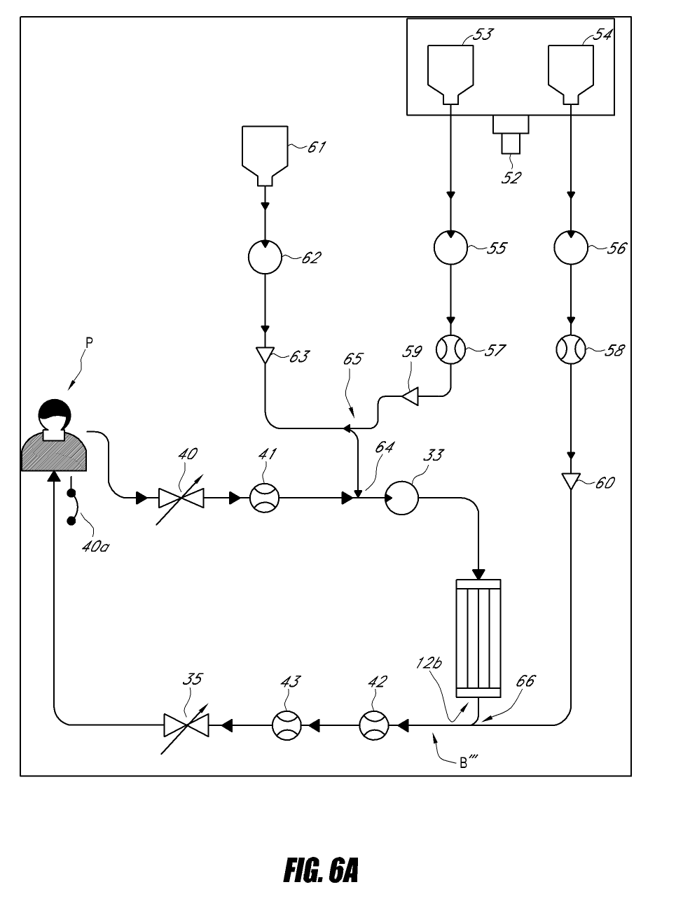

FIG. 5 shows one embodiment of a blood flow path B″ in a dual mode hemodialysis machine, such as the hemodialysis machine 100. In the illustrated embodiment, blood b is drawn from a patient via a needle connected to a conduit via a luer connector C. The blood flow b can flow past a flow meter 33c and pressure meter 33b, via a pump 33 and pressure sensor 33a into a blood inlet of the dialyzer 12. The dialyzer 12 removes waste and impurities from the blood b and purified blood b′ exits the dialyzer 12 and then passes through a bubble detector 34, flow meter 34a and pressure meter 34b, past a venous clamp 35 and is then returned to the patient. In the illustrated embodiment, luer connectors C′ are connected via a check valve 33d to the blood flow path B″, where the luer connectors C′ can be used deliver other substances (e.g., medication) to the blood b′ before passing the blood b through the dialyzer 12, as further discussed below.

FIG. 6A shows another embodiment of a blood flow path B″′ in a hemodialysis machine, such as the hemodialysis machine 100. In the illustrated embodiment, blood b can be drawn from the patient P past an arterial clamp 40 and flow sensor 41 by a pump 33, such as a peristaltic blood pump. The blood flow b then passes through the blood flow inlet 12a of the dialyzer 12. The blood flow b′ exits the dialyzer 12 via the blood flow outlet 12b once impurities have been removed from the blood b′. The blood flow b′ the passes through a bubble detector 42, and past a flow sensor 43 and venous clamp 35, before being returned to the patient P. The arterial clamp 40 and venous clamp 35 can be operated as is known in the art to stop or allow flow of blood b, b′ along the flow path In other embodiments, the arterial clamp 40 can be omitted. Additionally, in one embodiment a venous leak detector 40a can be provided to detect blood leaks in the return path.

With continued reference to FIG. 6A, in one embodiment, a fluid (e.g., saline bolus) can be injected from a container 53 via a pump 55 (e.g., pre-dialyzer infusion pump), past a flow sensor 57 and heck valve 59 to a location on the blood flow path B″′ upstream of the blood pump 33. In another embodiment, a fluid (e.g., saline) can be injected from a container 54 via a pump 56 (e.g., post-dialyzer infusion pump), past a flow sensor 58 and check valve 60 into a location on the blood flow path B″′ downstream-of the blood flow outlet 12b of the dialyzer 12. The containers 53, 54 can be disposed on a heated or warming tray 52 to maintain the fluid in a desired temperature range. The warming tray 52 can be separate or integral to the pumps 55, 56 to pre-warm the fluid. The hemodialysis machine 100 can include a control module that controls the operation of the warming tray 52. In another embodiment, the control module can be separate from the hemodialysis machine 100. In still another embodiment, Heparin can be injected from a container 61 via a pump 62 (e.g., Heparin pump) and check valve 63 to a location on the blood flow path B″′ upstream of the blood pump 33 for anticoagulation of the blood flowing along the blood flow path However, in other embodiments, one or more of the Heparin injection, pre-dialyzer fluid infusion and post-dialyzer fluid infusion can be omitted.

In one embodiment, as illustrated in FIG. 6B, the pump 55 can be operated to inject a rinse (e.g., cleaning fluid) from the container 53 to the blood flow path B″′ when the flow path is disconnected from the patient P, in order to rinse (e.g., clean) the blood flow path and allow for the dialyzer 12 to be used more than once.

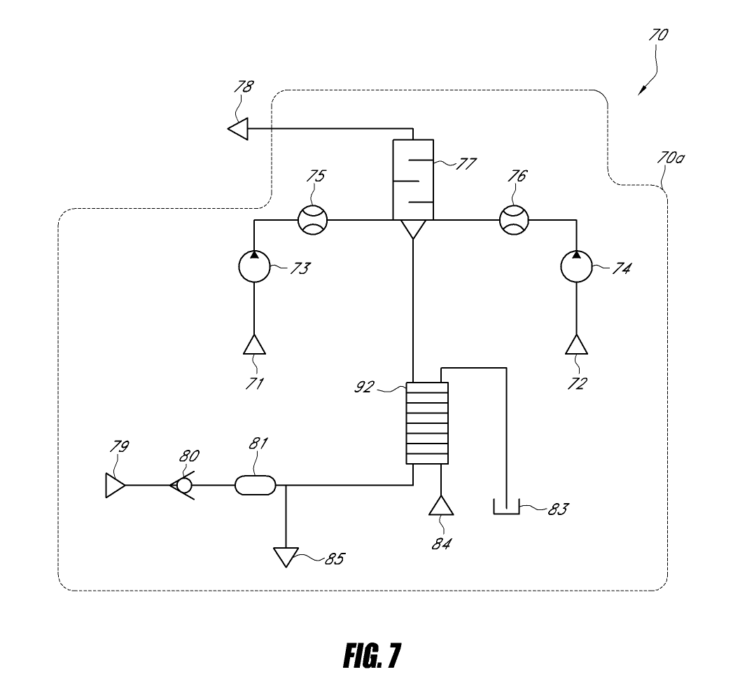

FIG. 7 shows one embodiment of a single-pass module 70 for use with a dual mode hemodialysis machine, such as the hemodialysis machine 100. In the illustrated embodiment, an acidic mixture (e.g., acetate) is pumped from a container (not shown) removably coupled to a connector 71 by a pump 73 and past a flow meter 75 into a mixing chamber 77. Additionally, a base mixture (e.g., bicarbonate) is pumped from a container (not shown) removably coupled to a connector 72 by a pump 74 and past a flow meter 76 into the mixing chamber 77. In the illustrated embodiment, water flows from a water source (not shown) and through a water treatments system (not shown), such as a reverse osmosis system (not shown) removably coupled to a connector 79, through a check valve 80 and pressure regulator 81. The water then flows through a heat exchanger 82, where the water is warmed (e.g., heated to a desired temperature range) before flowing into the mixing chamber 77, where the water mixes with the acidic and base mixtures. A heated fluid flows through the heat exchanger 82 via a connector 84, where it transfers heat to the water flowing through the heat exchanger 82, and then flows to a drain 83. The single-pass module 70 can be housed in a housing or cartridge 70a, and can be removably coupled to the dialysate flow path of the hemodialysis machine 100, so the hemodialysis machine 100 can operate in single-pass mode. The single-pass module 70 can be coupled to a dialysate flow path D of the hemodialysis machine 100 via a connector 78 that can connect to the flow path D upstream of the dialyzer 12.

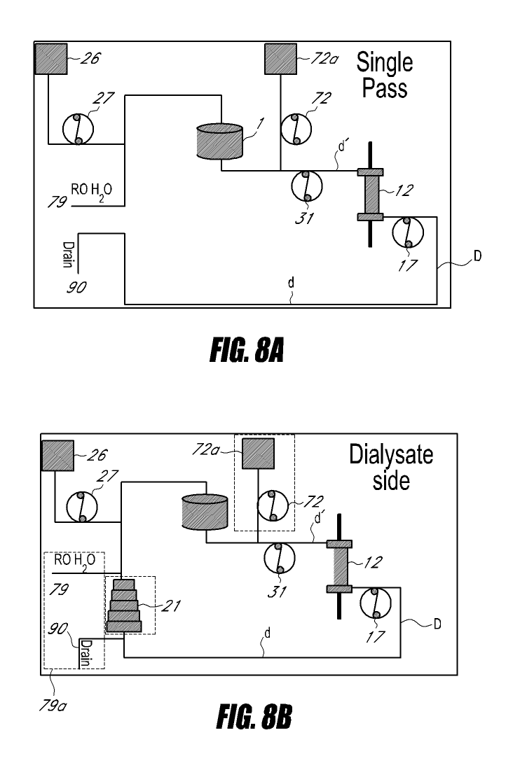

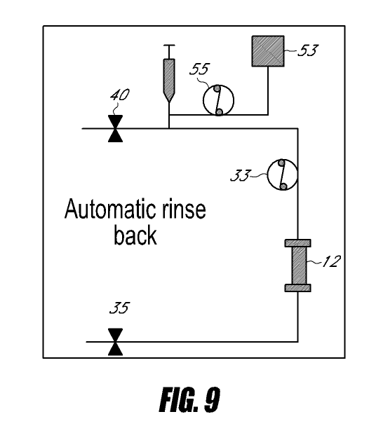

In one embodiment, a single-pass module can be incorporated into a dual mode hemodialysis machine having a sorbent mode system, as shown in FIG. 8A. In the illustrated embodiment, the pump 27 (e.g., infusate pump) can be used to inject acetate (or bicarbonate) from the container 26 into the clean dialysate flow path Purified water is provided from a water treatment system (not shown) via a connecting line into the dialysate flow path d′, where it mixes with the acetate (or bicarbonate) delivered by the pump 27. The mixture then flows to the chamber 1. Pump 72 injects bicarbonate (or acetate) from a container 72a into the dialysate flow path d′ for mixing with the purified water and acetate (or bicarbonate). The dialysate d′ then flows into the dialyzer 12. From the dialyzer 12, the unclean dialysate d flows to a drain 90 for disposal.

As shown in FIG. 8B, a dual mode hemodialysis machine, such as the hemodialysis machine 100, can optionally operate in a sorbent mode or single-pass mode. In the sorbent mode, the pump 72 is isolated from the dialysate flow path D, for example by a valve. Similarly, a single-pass inflow and outflow unit 79a, which can include the connection 79 to the water treatment system and to the drain 90, can be isolated from the dialysate flow path D via valves. In such a configuration, the used dialysate d can flow through the sorbent cartridge 21 and return to the dialysate inlet of the dialyzer 12 via the chamber 1 and pump 31, as discussed above. In the single-pass mode, isolation valves (not shown) can be actuated to isolate the sorbent cartridge 21 from the dialysate flow path D, and valves can actuated to place the pump 72 and single-pass inflow/outflow unit 79a in fluid communication with the dialysate flow path D, thereby allowing the dialysate flow path D to operate in single-pass mode, as described above. Advantageously, the dual mode hemodialysis machine 100 can be used in single-pass mode in facilities that have a dedicated water source and water treatment systems (e.g., a hospital), as well as in sorbent mode when the machine 100 is used in remote locations or while traveling, where a dedicated source of water or water treatment system are not available, as well as where water may be scarce.

In one embodiment, a hemodialysis machine, such as the hemodialysis machine 100, can be operated in automatic rinse-back mode to automatically return blood to the patient at the end of a dialysis treatment or upon detection of a irrecoverable error in the system (e.g., loss of power), as shown in FIG. 9. Once it is determined that rinse-back is required (e.g., end of a treatment, or due to a designated alarm), the hemodialysis machine can be operated to perform one or more of the following steps. The saline reservoir pump 55 starts. The arterial clamp 40 closes, saline now flows through the dialyzer 12, emptying it of the blood contained therein, which may be between about 2 cc to about 300 cc. After a predetermined volume of saline has been introduced, the blood pump 33 stops (e.g., to inhibit further flow into the dialyzer 12) and the arterial clamp 40 opens, allowing saline to back flush the arterial line of blood. In one embodiment, the saline pump 55 may stop briefly during the change-over to prevent an over-pressure in the fluid lines. After the arterial line is flushed (e.g., using a fixed volume), the system 100 may stop or return to flushing the dialyzer 12. The patient lines can safely be removed once the blood has been returned to the patient. Advantageously, the machine 100 can be operated to restore the maximum amount of blood to the patient (e.g., from the dialyzer 12) in the event of an alarm or detected problem or error in the operation of the machine 100.

In one embodiment, the hemodialysis machine 100 can have clinic and home/nocturnal settings, where a treatment can be set for a predetermined time/UF amount, etc. At the end of the prescribed treatment, the system 100 rinses the lines (e.g., blood flow path B), returning the blood to the patient automatically. Advantageously, the hemodialysis machine 100 can operate to perform a dialysis treatment without the need of nurse/patient intervention. Once the treatment is completed, the lines are in a “safe” condition, empty of blood, with the pumps stopped.

In one embodiment, the hemodialysis machine 100 can include a leak detector that detects a fluid leak within the casing of a dialysis machine via a humidity sensor. In one embodiment, mounted inside the machine 100, a device is used to monitor humidity within the casing (e.g., in an exhaust of the machine 100). If a level of the sensed humidity rises during operation of the hemodialysis machine 100, a leak may be present. In one embodiment, an alarm (e.g., visual, sound) can activate if the sensed humidity level rises above a certain level.

In one embodiment, the hemodialysis machine 100 can include a vibrating dialyzer mounting to aid in the removal of air during priming of the blood lines of the machine 100. The base can be a standard dialyzer clamp. Embedded in the clamp can be a vibrating mechanism. In one embodiment, the vibration generated by the vibrating mechanism can be small in amplitude with a frequency of 100 s to 1000 s Hz. During priming, the vibrator can be activated for a short period (e.g., a few seconds to a few minutes) to loosen any residual air bubbles in the dialyzer 12. The vibrating mechanism, in one embodiment, can be operated by a controller, such as the controller described below.

The embodiments of the dual mode hemodialysis machine, such as the hemodialysis machine 100, discussed above can use a control module having a controller (not shown), such as a computer controller (e.g., having a processor, memory and communications module) to control the operation of the hemodialysis machine 100. In one embodiment, the controller controls the operation of the pumps, such as the pumps 17, 31, 33, 55, 56, 62, and can receive sensed information from the various flowrate and pressure sensors (e.g., flow rate sensors 16, 7a, 41, 43 and pressure sensors 8, 18) in the machine 100. For example, the controller can control the speed of operation of a pump, such as the pump 17, as discussed above. In one embodiment, a pump speed is input into the hemodialysis machine 100 (e.g., by a nurse, or user) at the start of a dialysis treatment. The controller can then control the operation (e.g., speed) of the pump 17 to increase or decrease the speed at which fluid is drawn from the patient via the dialyzer 12. In one embodiment, the control module can include one or more algorithms for operating the machine 100 (e.g., stored in the memory of the controller). Such algorithms can be used to operate the hemodialysis machine 100 during different scenarios, such as during an automatic rinse-back mode or a loss of power, based on sensed operation data (e.g., flowrate, pressure, power, humidity). The control module can also actuate alarms (e.g., visual, auditory) when there is an error (e.g., leak detected, loss of power) in the operation of the hemodialysis machine 100. In one embodiment, the control module can include a communication module that can send and receive information, for example, wirelessly via an Rf transmitter.

Of course, the foregoing description is that of certain features, aspects and advantages of the present invention, to which various changes and modifications can be made without departing from the spirit and scope of the present invention. Moreover, the dual mode hemodialysis machine need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed dual mode hemodialysis machine.

What is claimed is:

1. A compact portable dual mode hemodialysis machine system, comprising:
a sorbent dialysis module comprising a sorbent cartridge configured to purify a dialysate fluid that flows therethrough, the sorbent dialysis module configured to return a purified dialysate fluid from the sorbent cartridge to an inlet of a dialyzer; and
a single-pass dialysis module comprising an acetate pump, a bicarbonate pump and a mixing chamber, the acetate and bicarbonate pumps configured to flow an acetate and bicarbonate into the mixing chamber, the single-pass dialysis module further configured to receive a desired amount of water from a reverse osmosis device, the single-pass dialysis module configured to direct used dialysate from the dialyzer to a drain,
wherein the single-pass dialysis module is configured to replace the sorbent cartridge to switch the operation of the dual mode hemodialysis machine system from a sorbent dialysis mode to a single-pass dialysis mode.

2. The system of claim 1, further comprising a blood pump configured to pump blood through the dialyzer at a flow rate of between about 50-600 mL/min and a dialysate pump configured to pump a dialysate through the dialyzer at a flowrate of between about 50-600 mL/min.

3. The system of claim 2, wherein the blood pump is configured to pump blood through the dialyzer at a flowrate of between about 50-450 mL/min and the dialysate pump is configured to pump the dialysate at a flowrate of between about 250-450 mL/min.

4. The system of claim 1, wherein the hemodialysis machine is configured to operate for a period of about 30 minutes following a power outage.

5. The system of claim 1, wherein the hemodialysis machine is configured to operate at a voltage of 100-240 V and less than about 1200 Watts.

6. The system of claim 1, wherein the hemodialysis machine is configured to perform a dialysis treatment in said sorbent dialysis mode using no more than about 6 liters of tap water.

7. The system of claim 1, wherein the sorbent cartridge allows a flow of up to about 450 mL/min therethrough.

8. The system of claim 5, wherein the sorbent cartridge has a maximum pressure drop of about 1900 mm Hg when dialysate flows therethrough at said flow rate of about 450 mL/min.
